(19) United States
(12) Patent Application Publication
(10) Pub. No.: US 2023/0346575 A1
(43) Pub. Date: Nov. 2, 2023

(54) AUTOMATED ANTHROPOMETRY MEASUREMENT SYSTEM FOR DESIGNING IMPROVED PROSTHETICS

(71) Applicant: QATAR UNIVERSITY, Doha (QA)

(72) Inventors: John-John CABIBIHAN, The Pearl (QA); Aya GABALLA, The Pearl (QA); Mohammed MUDASSIR, Doha (QA)

(21) Appl. No.: 18/110,853

(22) Filed: Feb. 16, 2023

Related U.S. Application Data
(60) Provisional application No. 63/310,711, filed on Feb. 16, 2022.

(51) Int. Cl. A61F 2/58; G06T 17/00; G06T 7/60; G06T 7/73

(52) U.S. Cl. CPC A61F 2/586; G06T 17/00; G06T 7/60; G06T 7/73; G06T 2207/10028; G06T 2210/56; G06T 2207/30052; G06T 2210/41; A61F 2002/5096

(57) ABSTRACT

An automated measurement system and method for acquiring human hand measurements for the automated construction of artificial hands may be provided. In some embodiments, the method for acquiring human hand measurements for the automated construction of artificial hands includes receiving, from one or more measurement devices, one or more scans of a hand of a subject. The method may also include identifying a plurality of landmark positions on the hand by performing an imaging analysis of the one or more scans, and determining a plurality of measurements related to one or more of a plurality of fingers of the hand based on one or more of the identified landmark positions. The method may generate a point cloud of the plurality of determined measurements.

### CROSS-REFERENCE TO RELATED APPLICATION

This application claims a priority benefit to U.S. Provisional Application No. 63/310,711, entitled “Development Of An Automated Measurement System For Acquiring Human Hand Measurements For The Automated Construction Of Artificial Hand,” filed on Feb. 16, 2022, which is incorporated by reference in its entirety.

### TECHNICAL FIELD

This invention relates generally to human prosthetic devices and anthropometry, and specifically, to the use of an automated measurement system to obtain accurate human hand measurements for developing life-like prosthetic devices, such as a hand, a finger, or a portion thereof.

### BACKGROUND

Anthropometry is the scientific field concerned with studying measurements of the human body. Measurements related to hand anthropometry has been used for various applications, including in medical science, fashion industry, and augmented/virtual reality applications. For example, in medical practices, a physical dimension of the hand and/or the wrist may be indicators of skeletal maturity and age of the human. As another example, hand anthropometry may have been used to determine the identity of a person, such as, for example, in situations where such evidence may be otherwise missing or falsified, e.g., in cases of human trafficking. As a further example, hand anthropometry may be used to study the interaction of persons with tools, machines, vehicles, and personal protective equipment—especially to determine the degree of protection against dangerous exposures, whether chronic or acute.

### SUMMARY

Various exemplary embodiments may provide an apparatus including at least one processor and at least one memory storing instructions. The stored instructions, when executed by the at least one processor, may cause the apparatus at least to receive, from one or more measurement devices, one or more scans of a hand of a subject, and identify a plurality of landmark positions on the hand by performing an imaging analysis of the one or more scans. The apparatus may also be caused to determine a plurality of measurements related to one or more of a plurality of fingers of the hand based on one or more of the identified landmark positions, and to generate a point cloud of the plurality of determined measurements.

Certain exemplary embodiments may provide a method including receiving, from one or more measurement devices, one or more scans of a hand of a subject, and identifying a plurality of landmark positions on the hand by performing an imaging analysis of the one or more scans. The method may also include determining a plurality of measurements related to one or more of a plurality of fingers of the hand based on one or more of the identified landmark positions, and generating a point cloud of the plurality of determined measurements.

Some exemplary embodiments may provide an apparatus including body frame of a prosthetic hand and a plurality of finger frames connected to the body frame. Each of the plurality of finger frames may be formed of multiple segments connected by ball and socket joints. The plurality of finger frames may be configured to bend and rotate relative to the joints and to maintain a position in which the finger frames are positioned.

### BRIEF DESCRIPTION OF THE DRAWINGS

For proper understanding of the features of some exemplary embodiments, reference should be made to the accompanying drawings, as follows:

FIGS. 1(a)-(g) illustrate examples of a human hand in different positions and the relative joint centers and landmarks;

FIG. 2(a) illustrates an example of a human hand depicting geometric measurement acquisition of finger lengths using finger segmentation and joint center locations from a palmar side view;

FIG. 2(b) illustrates an example of a human hand depicting geometric measurement acquisition of finger lengths using finger segmentation and joint center locations from a lateral side view;

FIG. 3(a) illustrates an example of a human hand depicting geometric measurements for determining surface palm length;

FIG. 3(b) illustrates an example of a human hand depicting geometric measurements for determining central palm length;

FIG. 4(a) illustrates an example of an experimental set-up from a top view;

FIG. 4(b) illustrates an example of an experimental set-up from a side view;

FIG. 5 illustrates an example of a three-dimensional (3D) model of a parametric prosthetic hand;

FIG. 6 illustrates an example of various anthropometric features of a hand;

FIG. 7 illustrates an example of a ball-and-socket joints having a low tolerance;

FIG. 8 illustrates an example of multiple variations of a 3D hand model generated by changing the anthropometric features;

FIG. 9(A) illustrates an example of a diagram showing a variety of grasping positions of a human hand by changing the anthropometric features;

FIG. 9(B) is a continuation of FIG. (A), and illustrates an example of a diagram showing a variety of grasping positions of a human hand by changing the anthropometric features;

FIG. 10 illustrates an example of electric powered prosthetic hands;

FIG. 11 illustrates an example of body-powered 3D-printed prosthetic hand;

FIG. 12 illustrates an example of a silicone-based static passive prosthetic hand;

FIG. 13 illustrates an example of a body-powered non-anthropomorphic upper-limb prosthesis;

FIG. 14(a) illustrates an example of a human hand depicting landmark mapping for scans used in an experiment herein;

FIG. 14(b) illustrates another example of a human hand depicting landmark mapping for scans used in an experiment herein;

FIG. 15 illustrates an example of a human hand depicting a joint center approach used for a computed tomography (CT) scan;

FIG. 16 illustrates an example of a prosthetic hand printed with polylactic acid (PLA) filament;

FIG. 17(A) illustrates an example of a diagram showing a variety of grasping positions performed by a prosthetic;

FIG. 17(B) is a continuation of FIG. 17(A), and illustrates an example of a diagram showing a variety of grasping positions performed by a prosthetic;

FIG. 18 illustrates examples of gestures performed by a prosthetic hand;

FIG. 19 illustrates an example of a diagram showing experimental results of a variety of measurements for different participants/subjects; and

FIG. 20 illustrates an example of an apparatus, according to various exemplary embodiments.

### DETAILED DESCRIPTION

It will be readily understood that the components of certain exemplary embodiments, as generally described and illustrated in the figures herein, may be arranged and designed in a wide variety of different configurations. The following is a detailed description of some exemplary embodiments of systems, methods, and/or apparatuses for the development of an automated measurement system for acquiring human hand measurements for the automated construction of artificial hands.

In anthropometry, considering hand shapes, sizes, and/or other measurements may be useful in the design of tools and devices, especially tools that may be custom-made or designed specifically for use by individuals of different demographics where anthropomorphic data may differ, such as different genders and/or ethnicities. Other applications may have identified distinctions of hand anthropometry. For example, the ratio between the second and fourth fingers, which may be known as the 2D:4D ratio, may be used as potential predictors of success in sports, musical ability, or as a biomarker for neurodevelopment disorders.

The measurement of non-injured hands may be performed for medical or cosmetic purposes, such as, for example, to develop a prosthetic hand or to purchase a cosmetic prosthetic glove for the missing hand. A computed tomography (CT) scan may generate measurement data of a non-injured hand, such as an amputee's non-injured hand, to produce, for example, a prosthetic cosmetic hand that may be similar in the size and shape to the non-injured hand. For example, CT scan data may be used to produce a prosthetic cosmetic hand that may have a 99.3% similarity to size and shape of the non-injured hand, albeit in mirror-image form.

Multiple procedures and/or techniques may be used, either alone or in combination, to obtain anthropomorphic measurements of a body part of a subject, such as a human hand. The measurements may be performed using direct and/or indirect methods. Non-limiting examples of the direct approach may involve the use of tools or devices, such as calipers and tape measures to obtain physical measurements from the subject. While this method offers simplicity, it can be riddled with inaccuracies due to incorrect placement of tools during the process and it also may be tedious and/or time consuming. This may be especially prevalent where multiple data points may be collected from a large number of subjects. It has been shown in 2D:4D studies that the measurements from photocopies were significantly lower than the direct measurements, which may be an effect of the shape of the pads at the fingertips being compressed during the photocopying.

Non-limiting examples of the indirect methods of measurement may include imaging techniques, such as photogrammetry, radiography, magnetic resonance imaging (MRI), and CT. A precision estimate for computer-based measurements may be higher than direct measurements using the caliper and the ruler. Radiography, MRI, and CT scans may have a higher accuracy and output resolution than the other methods. Radiography, MRI, and CT scans may measure a large number of data points on an object's surface and may represent the data points as a point cloud, which can be converted to a 3D model for anthropomorphic applications. The use of point clouds may increase the possibility of bulk and remote data collection and processing. These imaging devices may have a limited availability, such as in hospitals and/or specialized clinics, may be expensive, may require an expert for their operation, and may be inaccessible to many subjects, especially in low-resource communities.

Other methods of indirect measurement may rely on using a 3D scanning device to scan the human hand using more accessible imaging sensors that are available at a lower cost. Since 3D scanning devices may not be optimized for applications in anthropometry, the 3D scanning devices may fail to produce sufficiently high-quality image and/or data outputs that can be used for analysis.

Conventional measurement techniques may also need the scan(s) of a hand to be taken in an upright position, with the fingers outstretched. Due to the nature of these imaging devices, maintaining hand rigidity and stability in such a position throughout the capture process remains a challenge, which may lead to inaccurate measurements. With either direct or indirect methods, there may be trade-offs in data collection time, achieving accurate results, and obtaining a complete appearance of the hand without any obstruction. For the direct method (e.g. rulers, protractors), the hand that is being measured may have to be supported on a flat surface to achieve accurate distance measurements. For the indirect method (e.g. MRI, CT scan), fine details of the hand and the gaps between fingers can lead to missing data and/or holes at low resolutions if parts of the hand or fingers are occluded. Further, contact with a surface may be problematic because the skin tissue on the hand and fingers may be compliant and may cause deformation. Large displacements may occur even with minimal contact forces. For example, a 2 mm displacement can be experienced with a 1 N contact force. Thus, for indirect methods, the subject/patient may have to raise one or both arms at a distance from the scanning table to get the full shape of the hand without contact.

Acquisition of accurate anthropometric hand data from the human hand may be useful for designing tools and devices, and may ensure that user interfaces are user-friendly, effective, and more comfortable by maximizing contact area, improving hand posture, and reducing musculoskeletal injury. For example, tools that were designed with anthropometric data from one ethnicity and be used by someone with a different ethnicity may have significant differences in tool sizes. There may also differences between genders. For example, the 5^{th} percentile male is around the size of the 50^{th} percentile female. For mission-oriented tasks, the usage of the 50^{th} percentile data may have disadvantages as this may consider a smaller segment of the population and not majority of the users/patients.

Studies on a 2D:4D ratio have shown that there may be gender differences between the lengths of the second (index finger; 2^{nd} digit) and fourth fingers (ring finger; 4^{th} digit). The 2D:4D ratio may be less than 1 for most males and the 2D:4D ratio may be equal to or more than 1 for most females. For example, a higher serum testosterone and lower estrogen levels may lead to lower 2D:4D ratios.

Various exemplary embodiments may provide one or more apparatuses and/or methods to provide a convenient and efficient method for anthropometric hand measurements. Some exemplary embodiments may provide for all size ranges of users to be considered and afford a more convenient method to collect anthropometric measures of the hand. Certain exemplary embodiments may provide a pose-independent indirect measurement collection process that can use inexpensive, commercially available 3D scanning devices. Certain exemplary embodiments may use processes that rely on determining measurements at the joint-centers of the hand to increase the accuracy of the results produced as compared to commonly used surface measurements of the hand.

The conventional direct and/or indirect methods perform measurements from the palm's surface of the hand with all the digits (finger) outstretched. Certain exemplary embodiments may advantageously provide one or more indirect measurement procedures for taking measurements on a human hand, which may be invariant of the pose of the hand. A point cloud may be implemented to provide the ability to obtain the measurements from within the hand at the center of the joints, which may enable indirect measurement collection while utilizing inexpensive, commercially available 3D scanning devices.

Various exemplary embodiments may provide one or more indirect measurement procedures to scan and measure a human hand in its natural pose with the elbows of the arms being supported by a support surface. Using 3D scanning devices, some exemplary embodiments may provide one or more procedures to measure the joint centers in the hand from the surface point cloud of a scan by a scanning device. Certain exemplary embodiments may also provide various 3D scanning devices that may improve accuracy and shorten a duration of data acquisition. In an example of a CT scan of a human hand used as a reference standard due to a high image resolution provided by the CT device, certain exemplary embodiments may demonstrate the usability of the method with the exact part models of prosthetic gloves and the feasibility of an automated anthropometric measurement of the human hand.

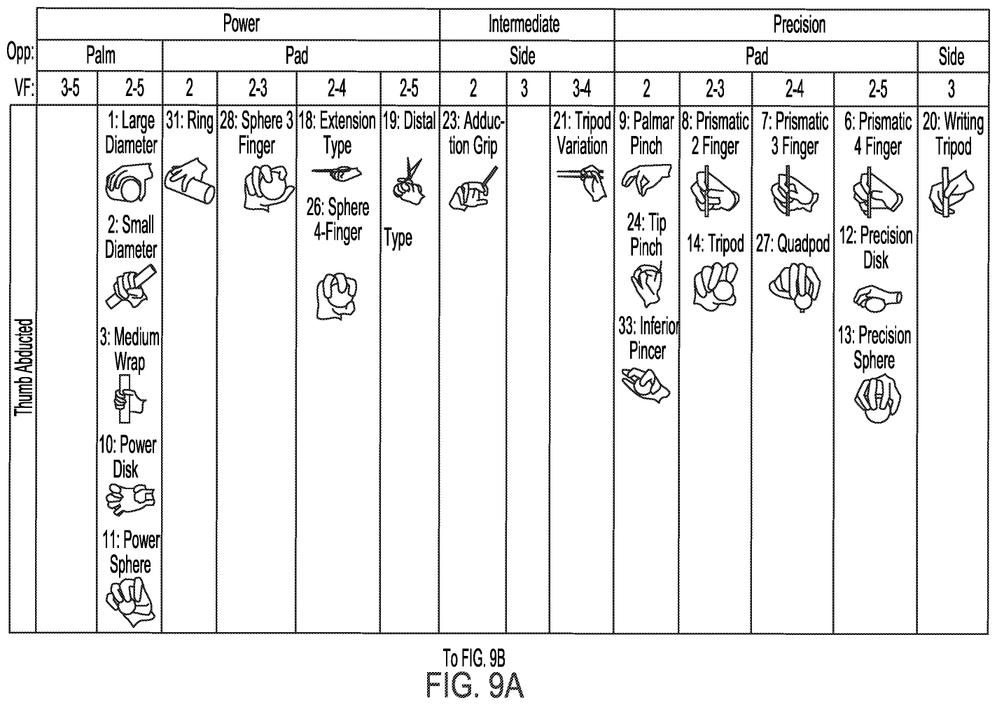

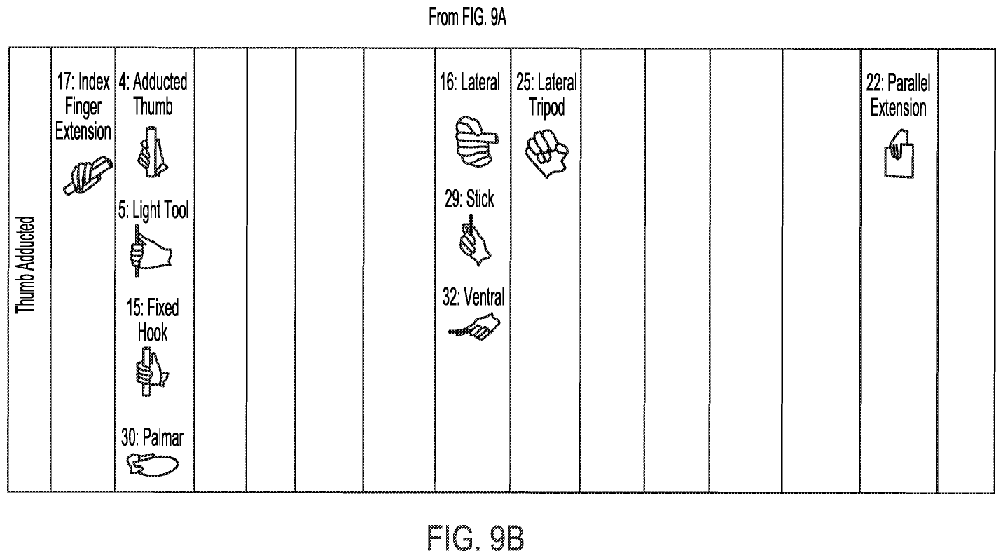

Measurements performed according to various exemplary embodiments may be used to develop a prosthetic device that exploits and innovates on the passive prosthesis model and adds grasping ability using dynamic ball-and-socket joints. According to certain exemplary embodiments, the prosthetic device may perform multiple grasping movements, such as, for example, 31 out of the 33 various grasps that a human hand may be able to perform, as shown in FIGS. 9(A) and 9(B). Non-limiting examples of the various grasps of the prosthetic hand may include grasping objects, such as credit cards, pens, coins, balls, bottles, plates, etc. Certain embodiments may provide a passive prosthetic hand that can perform these large number of grasps and may be designed or custom designed based on measured dimensions of the hand of the individual subjects/patients. The processes of generating the designs may be simplified by implementing a computerized analysis of 3D scans, which may use an algorithm, that uses as inputs the anthropometric features obtained from the 3D scans and outputs a customized 3D model of a prosthetic.

Performing measurements on a human hand to design prosthetics may require acquisition of large amounts of anthropometric hand data. Various exemplary embodiments may provide one or more procedures that may enable the extraction of required measurements in a relatively inexpensive and controlled manner. Certain embodiments may automate identification of natural landmarks through the implementation of computer vision techniques. This automated identification according to certain exemplary embodiments may facilitate research applications or the development of neural models to perform generalizations and predictions.

Various exemplary embodiments may provide a 3D printed parametric prosthetic hand that may provide value to various technologies and/or markets due to its ability to achieve a large number of grasps. Further, parametric modelling may be extended to include the forearm and the arm by establishing the mathematical relations between the hand, forearm, and arm. Some exemplary embodiments may also be extended to the body-powered and electric-powered prostheses. The embodiments discussed herein are not intended to limit the scope of the invention(s). The devices, procedures, methods, etc., discussed herein may be extend to various other body parts and/or using different materials, such as materials other than polylactic acid (PLA) or acrylonitrile butadiene styrene (ABS).

Certain techniques may rely on the quality of the 3D hand models obtained from 3D scanners and/or other imaging devices. Low quality models may provide results that may not be accurate because some exemplary embodiments may rely on visually (or computer recognized) identified natural landmarks that appear on the surface of the hand to locate joint centers and perform the calculations. If a 3D mesh obtained from, for example, a 3D scanning device, has been subjected to deformations due to significant motion during scanning, results may again be inaccurate.

Since the process may depend on visual landmark identification, it may be subject to errors. In cases where the procedure is performed manually, the individual performing it may need a small amount of practice in order to recognize the necessary landmarks, especially on a mesh of lower resolution. When the method may be performed automatically with the use of computer software, a well-trained model may be required to avoid the potential for misidentifying landmarks and resulting in calculation errors.

According to various exemplary embodiments, 3D printed products may be employed, and such products may use materials with a requisite quality and performance. A design fabricated using steel may last much longer than if the design were fabricated using polymeric materials, such as PLA or ABS. The prosthetics may be meant for lightweight tasks and social interactions, and the patient may need to have a healthy hand to operate this device. A glove may be worn over this prosthesis for cosmetic enhancements.

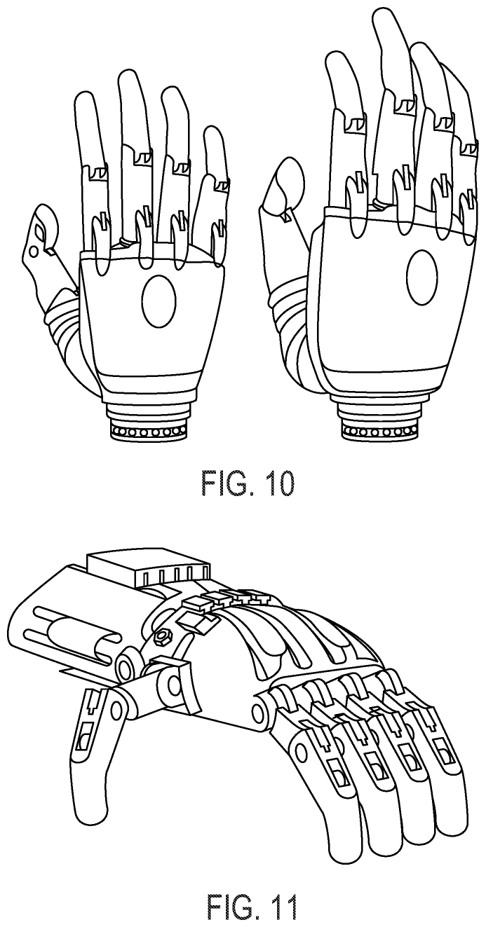

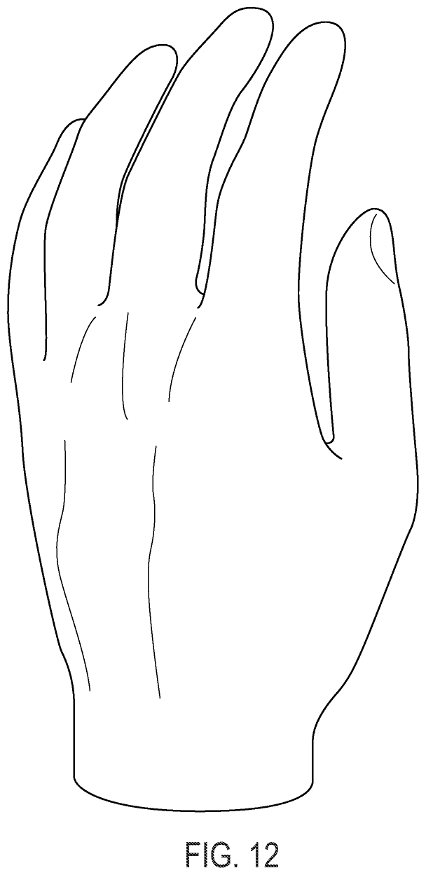

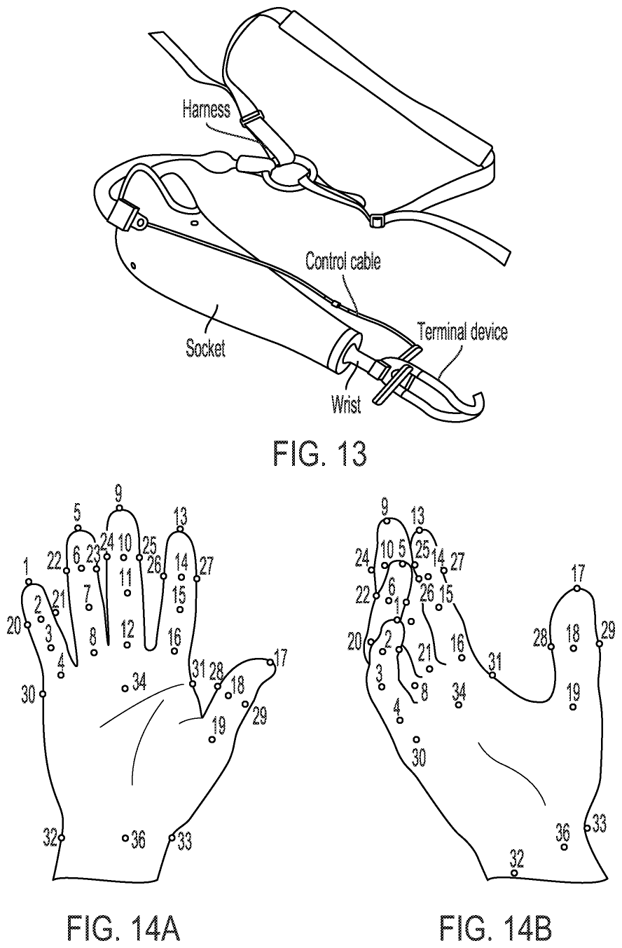

Prostheses may be categorized as body-powered, electric-powered, and passive. Electric-powered prostheses, as shown in FIG. 10, may offer many grasping features and functionalities, but may be expensive to buy and a number of grasps they can perform may be less than six. Open-source 3D-printed body-powered prostheses may be used, but may offer limited functionality and poor grasping performance. FIG. 11 shows an example of a prosthetic hand that can flex by non-elastic cords and extend by elastic cables. Further, life-like passive prosthetics may be more affordable and may reduce social stigma, but may not offer grasping functionality. An example of a life-like passive prosthetic may be shown in FIG. 12. FIG. 13 shows an example of a prosthetic device that may be non-anthropomorphic while offering some functionality for grasps and manipulations.

Various exemplary embodiments may provide a parametric prosthetic that may exploit and innovate on the passive prosthetic model. By using ball-and-socket joints, the passive device may achieve motion for the fingers and remain passive in that no active force is required to maintain the grasp once it is performed. Certain exemplary embodiments may be fabricated by 3D printing, which increases the accessibility and affordability. To reduce costs, time, and inconvenience for repairs, broken finger segments may be re-printed, instead of reprinting the complete hand again.

Certain exemplary embodiments allow for the determination of commonly used geometric measurements on the human hand. Various exemplary embodiments may provide detailed procedures/processes to identify landmark points on the human hand that correspond to a joint. To reduce inaccuracies by visually approximating the locations of the joint-centers, certain exemplary embodiments may provide a mathematical solution to derive their positions and calculate the corresponding measurements. Points on the surface of the hand may be identified based on their relevance to the calculations required to determine the joint-center locations and measurements. Since certain embodiments deal with the ellipsoid joints in the hand and wrist, joint-centers may be defined as the intersection of the semi-minor and semi-major axes of the joints.

Landmark points used in the calculations may be determined based on their ability to accurately reflect joint center locations. As a result, landmark points may be located at positions that are minimally affected by muscular flexion or extension. This may be because the thin muscle layers underneath the skin results in negligible dislocation during finger flexion. Such landmark placements may allow for the pose-invariant calculation of the chosen dimensions.

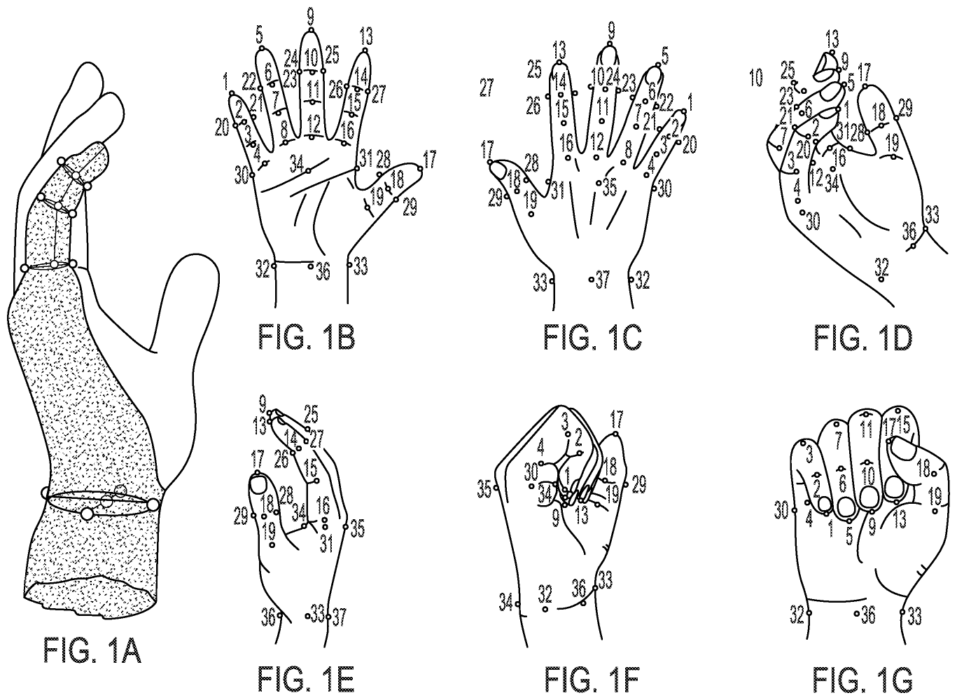

FIGS. 1(a)-(g) illustrate examples of landmark points that may be mapped and shown in various hand orientations. The points representing finger joint locations may be located at the center of the distal interphalangeal (DEP) joint, proximal interphalangeal (PIP) joint, and metacarpophalangeal (MCP) joint. For example, points 20-36 may represent opposing pairs lying on the finger surface on the same plane as the distal digital crease.

Point 30-31, 32-33, 34-35, and 36-37 may represent opposing pairs on the hand surface and may be used for calculations of palm and wrist circumference. For example, point 30 may be located on the side of the hand right below the little finger at the base of the proximal phalanx. An absence of a thick muscle layer at this location may prevent the landmark position from changing during flexion or extension. Point 31 may be located directly opposite point 30 at the tip of the proximal transverse crease. The direct line connecting points 30 and 31 may cut horizontally through the center of the hand and may represent the palm width. Point 34 may be located on the palm surface on the same horizontal plane as points 30 and 31 and may intersect the perpendicular bisector.

Point 34 may be identified by its convenient placement on the distal transverse crease. Point 35 may be located on the back of the hand directly opposite point 34 and slightly below the top of the third metacarpal bone.

According to certain exemplary embodiments, there may be three parameter categories that can be determined using the identified landmarks. The first category may include the measurements representing finger lengths, which rely on finger joint center landmarks. The second category may include the wrist and palm circumference measurements. The third category may be comprised of the palm length, which may be defined as the vertical distance between the center of the third MCP joint and the wrist center. Calculating these parameters may involve determining a number of secondary measurements, which may also be used later for anthropomorphic modelling and reconstruction. Exemplary calculations and measurement acquisition processes are described in detail herein.

For the first category that may include the measurements representing finger lengths, the semi-minor and semi-major axes of the interphalangeal joints may be observed to be approximately equal and treated as diameters. It may be assumed that the finger has a uniform cylindrical shape with a rounded top, or half capsule shape. In some exemplary embodiments, it may further be assumed that the finger has a uniform radius at any given horizontal plane.

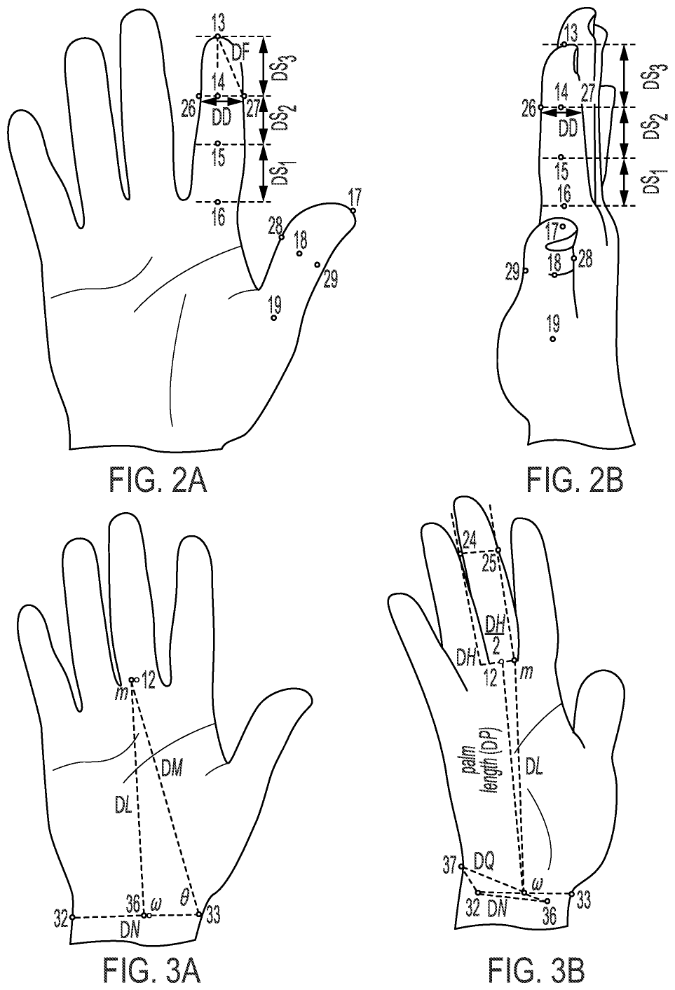

To achieve pose-invariance, various exemplary embodiments, as shown in FIG. 2(a) and FIG. 2(b), may measure finger lengths by segmenting the finger into 2-3 separate units between the joints. The lengths of those units were then measured separately. The measurements may be added to determine the total finger length. Determining the lengths of the proximal and medial phalanges may use the measurement of the distances between the corresponding points on the surface on the side of the finger, due to the negligible displacement effects of finger flexion on the points located at the side of the finger.

The total finger length may be calculated in Equation (1) by adding the lengths of all three segments:

ΔSₜₒₜₐₗ=ΔS₁+ΔS₂+ΔS₃ (1)

where the total finger length may be represented as ΔStotal and length of proximal, medial, and distal phalanges may be denoted as ΔS1, ΔS2, and ΔS3, respectively.

Due to the curved nature of the fingertip, further techniques or processes may be employed to determine the length of the distal phalanx. The finger diameter may be perpendicular to the length of the distal phalanx. As shown in FIG. 2(a) and FIG. 2(b), point 14 may correspond to the location of the distal interphalangeal (DIP) joint center at the midpoint of the finger diameter. The distance between points 13 and 27 may be the hypotenuse, and can be determined by measuring the distance between the fingertip and any point on the DIP crease.

The length of the distal phalanx may be calculated using Equation (2):

$$Δ {S}_{3} = \sqrt{{( Δ F )}^{2} - {( \frac{\text{?}}{2} )}^{2}} \qquad ( 2 )$$

$$\text{?} \text{indicates text missing or illegible when filed}$$

where ΔS3 may be the length of the distal phalanx, and the finger diameter and the hypotenuse may be denoted as ΔD and ΔF, respectively.

For the second category that may include wrist and palm circumference measurements, circumference calculations according to certain exemplary embodiments may use, for example, four secondary parameters, which are distances that may correspond to the opposing points present on the palm surface, as shown in FIG. 1. The palm and wrist circumference measurements may not form circular shapes and may be ellipses.

As a result, Ramanujan's equation for approximating the perimeter of an ellipse may be used, which is presented as Equation (3):

$$c \approx π ( Δ N + Δ Q ) ( 1 + \frac{3 {( \frac{Δ N - Δ Q}{Δ N + Δ Q} )}^{2}}{10 + \sqrt{4 - 3 {( \frac{Δ N - Δ Q}{Δ N + Δ Q} )}^{2}}} ) \qquad ( 3 )$$

where c, ΔN, and ΔQ may represent the circumference, the semi-major axis, and the semi-minor axis of the ellipse, respectively. When applied to the illustrations in FIG. 1, lines at points 34, 35, 36, and 37 may be shown to represent the minor axes, and lines 30-33 may represent the major axes.

For the third category that may be comprised of the palm length, certain exemplary embodiments may determine the central palm length, defined as the distance between the third MCP joint center and the wrist center. As shown in FIG. 3(a) and FIG. 3(b), point 12 may correspond to the third metacarpophalangeal (MCP) joint center while the midpoint of points 36 and 37, point ω, may correspond to the wrist center. The distance between the corresponding palm surface position of point 12, denoted as point ω, and point 33 can be measured, and may represent the palm hypotenuse. The angle between points 12, 33, and 32 may represent the wrist angle, and can also be determined from a scan using inspection and measurement tools.

The surface palm length between point μ and the midpoint, ω can be calculated using Equation (4) as follows:

$$Δ L = \sqrt{{( \frac{Δ N}{2} )}^{2} + Δ {M}^{2} - 2 Δ N Δ M cos ( θ )} \qquad ( 4 )$$

where ΔL may represent the surface palm length, and ΔM, ΔN, and θ may represent the palm hypotenuse, the semi-major axis of the wrist, and the wrist angle, respectively.

The diameter of the middle finger may then be determined by measuring the distance between points 24 and 25, as shown in FIG. 3(b). The line from the MCP joint center to the wrist center, ω, may form a right angle with the diameter line of the middle finger. As a result, we can use the calculated value to determine the central palm length using Equation (5):

$$Δ P = \sqrt{{( Δ L )}^{2} - {( \frac{Δ H}{2} )}^{2}} \qquad ( 5 )$$

where ΔP may represent the central palm length, and ΔL and ΔH may represent the surface palm length and the middle finger diameter, respectively.

In certain exemplary embodiments, a helical CT scanner, such as an Aquillion 64, Toshiba Medical Systems, Japan, may be used for reference with the following scan parameters: 120 kV, 150 mA, 75 mA exposure, 500 ms exposure time and 0.5 mm slice thickness. Four commercially available scanners, as shown below in Table I, may be used to obtain the measurements from a single human hand and may be compared to the imaging data from a CT scan. According to certain exemplary embodiments, the scanners in Table I may be selected based on their popularity among researchers as well as their accessibility on the global market. For each device, 10 scans per device may be taken from the participant.

TABLE 1
PROPERTIES OF THE 3D SCANNERS
USED IN THE EXPERIMENTS

| | | | | | Price |
|---|---|---|---|---|---|
| Scanner | Figure | Model Details | Resolution | | (approx. [symbol] |
| Creaform | [symbol] | GelScan20, [symbol] Inc., PA, USA | 0.1 | mm | $20,000 |
| Kinect | [symbol] | Kinect for Windows, | 5 | mm | $300 |
| | | Microsoft, WA, USA | | | |
| Sense | [symbol] | 2^{nd} Generation, 3D Systems, SC, USA | 1 | mm | $700 |
| Structure | [symbol] | ST01, [symbol] Inc., SF, USA | 0.5 | mm | $400 |

[symbol]

indicates data missing or illegible when filed

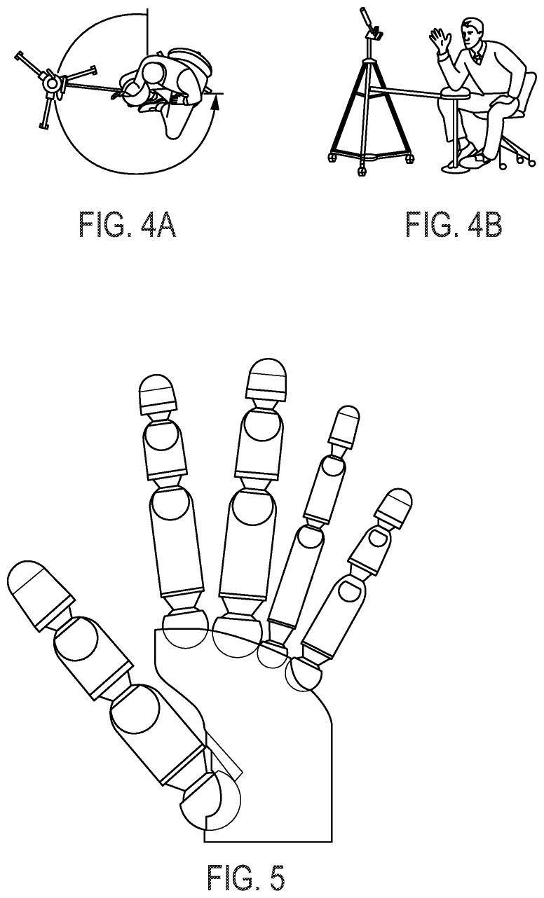

FIG. 4(a) and FIG. 4(b) illustrate an example of an experimental set-up for scanning, which may not require the acquisition of any defining features of the participant and may output a mesh of the subject's hand. During the experiment, the subject may raise their arm at a distance from the scanning table to get the full shape of the hand without contact. To obtain accurate scans of the human body in an unconstrained or semi-constrained manner, various exemplary embodiments may provide a scanning apparatus that may limit the hand movements to obtain, for example, distance and circumference measurements. To make the participant comfortable, an armrest may be provided upon which the arm is placed, which is shown in FIG. 4(a) and FIG. 4(b).

The scanner may be mounted on a tripod that is connected to the armrest with an adaptor to limit the degrees of movement during the tripod's rotation. This will ensure that the arm remains within the scanning frame throughout the process and may help reduce artifacts in the captured scans. Multiple adaptors may be designed to mount the scanners to the tripod. The full scan of the hand can be obtained with a 270° rotation of the tripod, where the scanner is mounted. All measurements may be obtained digitally using computer-aided design software, such as, for example, Meshlab and Fusion 360, Autodesk, etc. Open-source image visualization software, such as 3DSlicer, may be used to convert the CT scan DICOM files to STL mesh files.

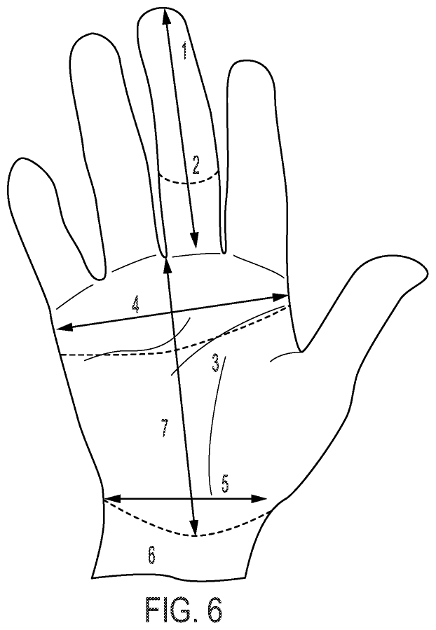

As shown in FIG. 5, various exemplary embodiments may provide a passive anthropomorphic prosthetic hand that may be formed of a palm and five fingers. Each of the fingers may have three ball-and-socket joints, which are located at proximal, middle, and distal locations. The proportions of finger segments may be similar to those of the human hand. The ball-and-socket joints may allow three degrees of freedom: rotation along x-axis, rotation along y-axis, and rotation along z-axis.

As illustrated in FIG. 6, seven anthropometric features may be used for generating the 3D model, which may include: 1) middle finger length, 2) middle finger circumference, 3) metacarpal circumference, 4) metacarpal diameter, 5) wrist diameter, 6) wrist circumference, and 7) palm length. The anthropometric features can be measured manually by a ruler and measuring tape or by 3D scanning the hand first and then post-processing for better accuracy. Mathematical relations may be established between the seven anthropometric features and the remaining parts of the hand. Since this design has no actuators, the motion may be performed by the healthy hand.

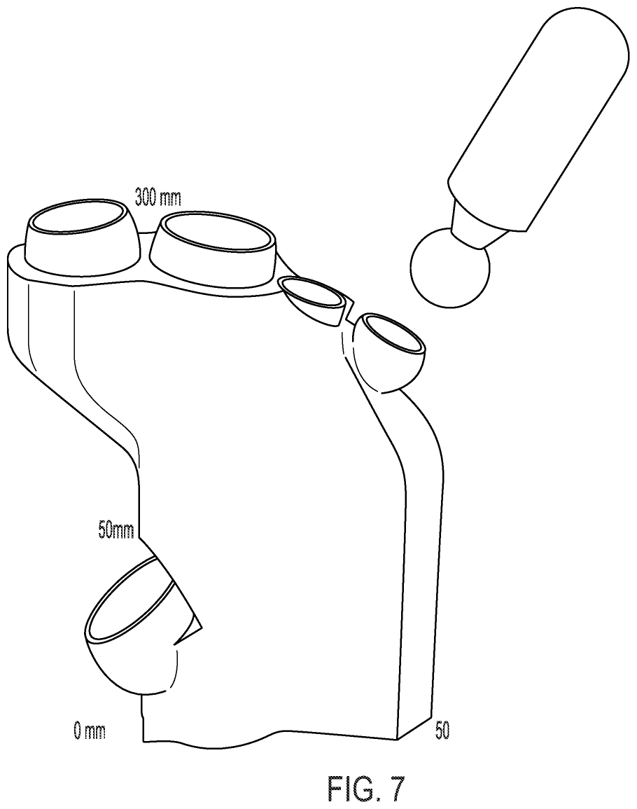

FIG. 7 illustrates an example of an exemplary prosthetic hand according to various exemplary embodiments. The prosthetic hand 700 may be formed a hand frame 710 and a plurality of finger frames 720. The plurality of finger frames 720 may be connected to the body frame 710 by a ball and socket joint 730. Each of the plurality of finger frames 720 may be formed of multiple segments (740, 750, 760) connected by ball and socket joints 730. The multiple segments may be a proximal segment 740, a middle segment 750, and a distal segment 760. A low-tolerance for the ball-and-socket joints may be required to prevent slippage and loose joints. The finger frames 720 may be configured to bend and rotate relative to the joints 730 and to maintain a position in which the finger frames 720 are positioned to facilities grasping. The body frame 710, fingers 720, finger segments 740, 750, 760, and joint 730 may be fabricated using 3D printing by using materials such as PLA and ABS. The exemplary ball joints may be inserted by force into the sockets as shown in FIG. 7. Friction may be the main force keeping the balls stable in the sockets.

Certain exemplary embodiments may provide one or more joint center-based procedures for determining pose-independent measurements from a human hand using an indirect approach. In some exemplary embodiments, the procedure may use a number of natural landmarks on the hand in order to localize the joint centers and complete the calculations. While specific calculations may explicitly detailed, additional measurements may be extrapolated using this data. For example, in certain exemplary embodiments, finger circumference may be a measurement that is used in custom tool manufacturing and for purchasing gloves. Although that calculation is not described, it can be determined from finger diameter using the formula ad, which was identified during calculations pertaining to the finger length.

Various modifications may be made to the exemplary procedures as well as other similar processes that use the joint-center approach to identify anthropometric data from the human hand.

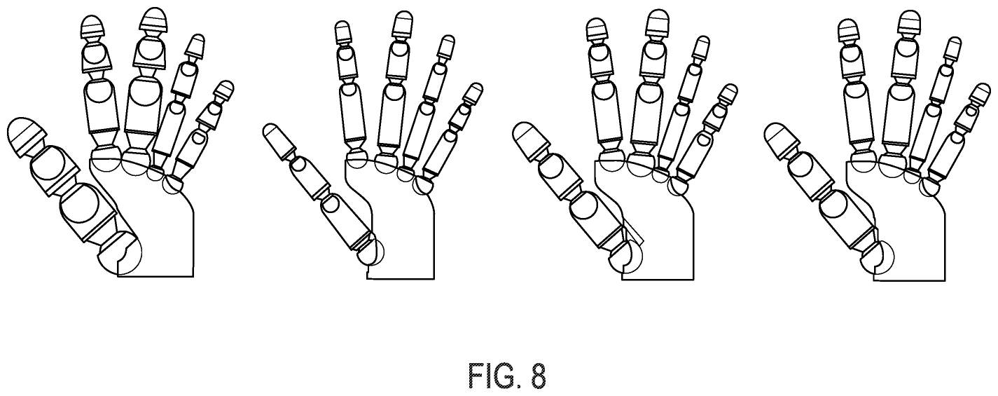

In certain exemplary embodiments, variations of a printed design may be possible through making changes to the anthropometric parameters because each patient may have their own set of hand features. FIG. 8 illustrates some examples of the parametric variations. It may be noted that there may be variations in the finger segments and the palm contours.

To determine whether the measurements obtained from the tested devices may be statistically similar to the measurements obtained from the CT scan, the data may be analyzed using a variety of exemplary methods. For example, a multivariate normality test may be used. The Henze-Zirkler values may be calculated to verify whether the data conforms to the assumptions of normal distribution. Once normality is confirmed, Hotelling's t-squared test may be used to compare all the collected data sets with the CT scan data to minimize the error resulting from performing multiple individual tests. This may allow the total error to be limited, such as to 5%. The devices that may have no significant difference from the CT scanner's measurements may be tabulated. Table II illustrates the results of an exemplary test using this procedure in which all statistical calculations were performed using RStudio (v1.2.5001, RStudio, Inc., USA).

TABLE II
CT scan reference measures (in mm) by parameters.

| Parameter | Measurement (mm) |
|---|---|
| Thumb | 60.55 |
| Index finger | 73.02 |
| Middle finger | 82.06 |
| Ring finger | 75.29 |
| Little finger | 58.17 |
| Palm length | 112.08 |
| Wrist circumference | 171.30 |
| Palm circumference | 201.50 |

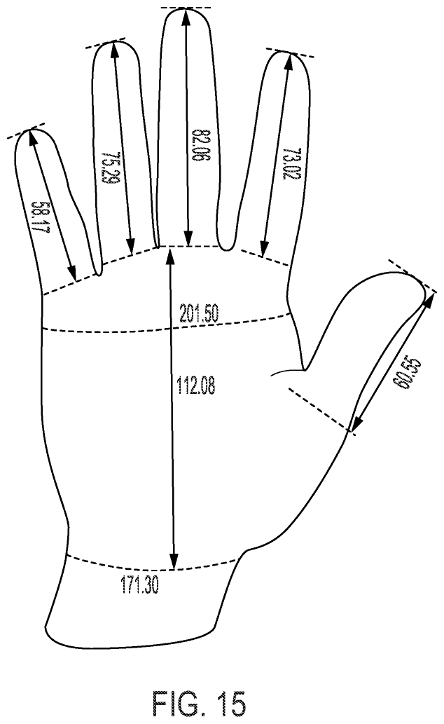

As shown in FIG. 14(a) and FIG. 14(b), the hand may be scanned with the CT scanner and 4 types of 3D scanners. The required parameters may be measured as described herein. FIG. 15 illustrates reference measures obtained from the CT scan which may be used for comparison purposes, such as in Table II.

The mean values of the results from all tested sample scans may be calculated and sorted by parameter. Prior to the analysis, a multivariate normality test may be conducted to verify the normal distribution, as shown below in Table III. The test showed that the data collected from all four devices fulfilled the normality assumptions.

TABLE III
Henze-Zinkler (HZ) coefficients and p-values of the devices.

| Scanner | Henze-Zirkler Coeff | p-value |
|---|---|---|
| Creaform | 0.934 | 0.103 |
| Kinect | 0.942 | 0.061 |
| Sense | 0.940 | 0.068 |
| Structure | 0.934 | 0.090 |

With the assumption of normality verified, it may be possible to proceed with the comparison. In order to analyze the 3D scan data, Hotelling's t-squared test may be applied to test the measurement similarity to the CT scan standard, as shown in Table IV. The results showed that three of the four scanners showed no significant differences as compared to the CT scan.

TABLE IV
statisical results for each scanner.

| Scanner | Hotelling's T² | df₁, df₂ | p-value |
|---|---|---|---|
| Creaform | 2.627 | 8, 3 | 0.230 |
| Kinect | 20.644 | 8, 2 | 0.047 |
| Sense | 9.763 | 8, 2 | 0.096 |
| Structure | 5.385 | 8, 2 | 0.166 |

To determine the accuracy relative to the CT scan measurements, for each of the scanners that conformed with the standard, the average standard error of the mean (SEM) may be calculated across all the measurement variables to account for the variance and the mean differences. These values can therefore be used to quantify the accuracy in comparison to the standard, and to one other.

Separate standard error calculations may be conducted for each variable and may be averaged per scanner to determine the relative standard error (RSE). According to the results shown in Table V, the Creaform scanner had the highest accuracy with respect to the CT scan data, followed by the Structure and the Sense scanner.

TABLE V
3D scanners’ measurement accuracy

| | Std Error of | Relative Std |
|---|---|---|
| Scanner | the Mean (mm) | Error (%) |
| Creaform | 1.27 | 98.54 |
| Structure | 1.34 | 98.61 |
| Sense | 1.80 | 98.12 |

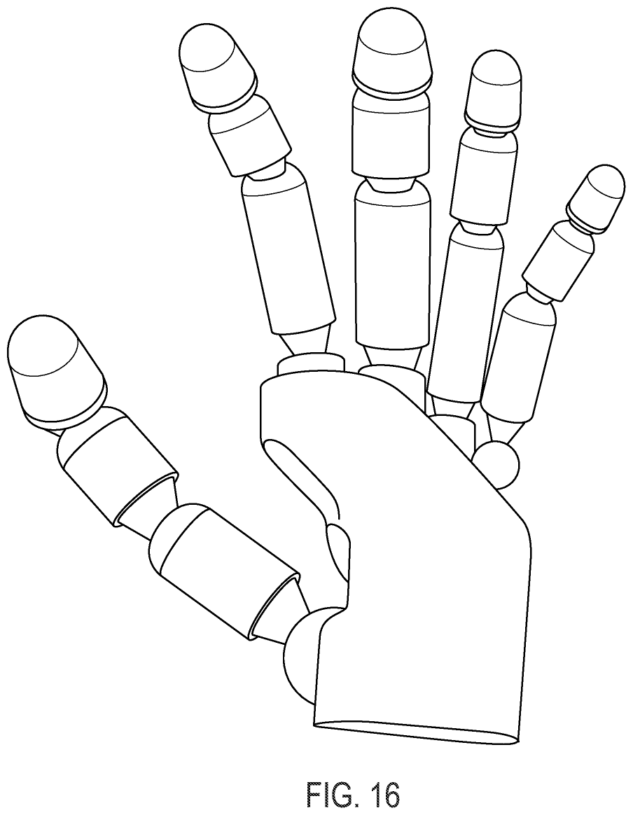

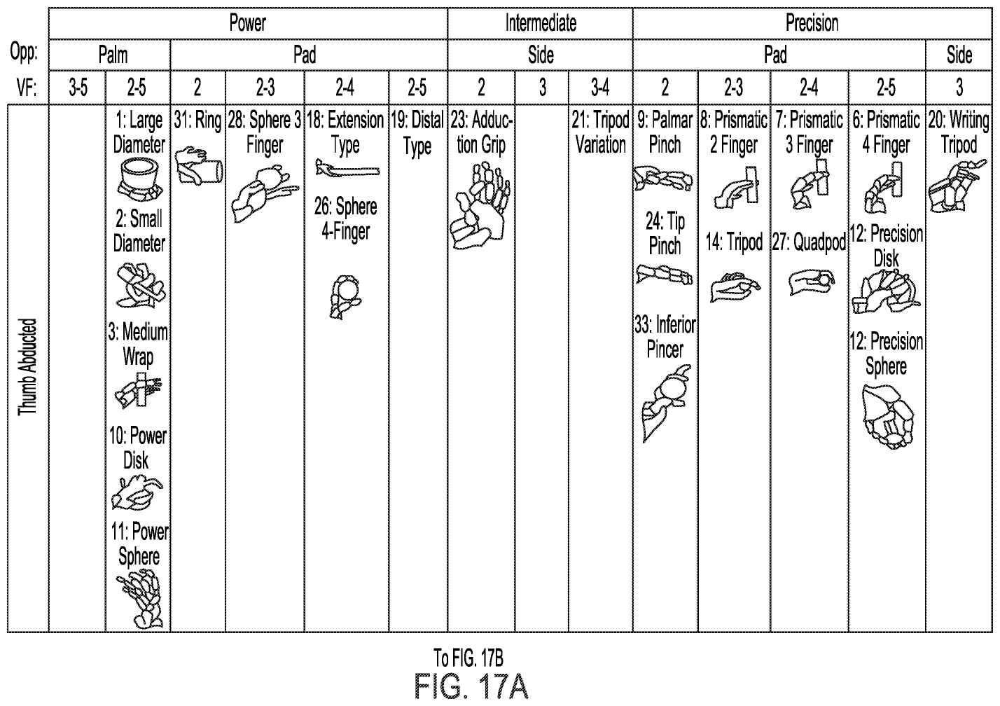

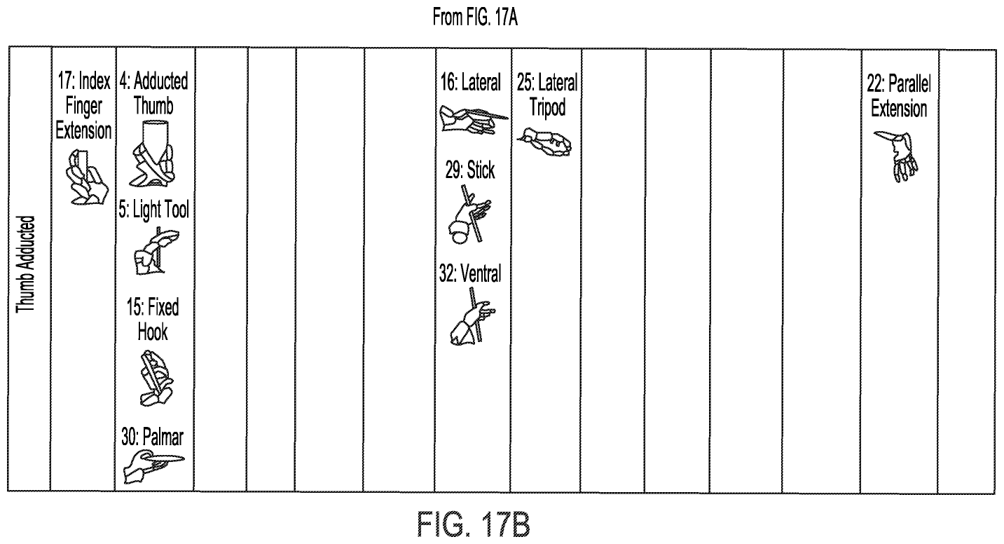

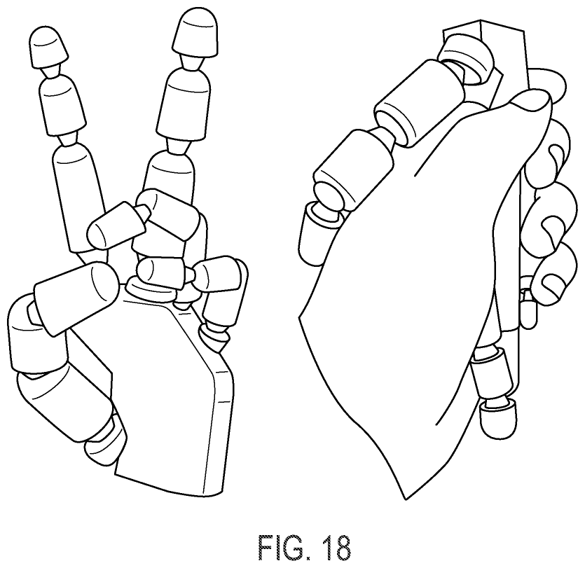

FIG. 16 illustrates an example of a prosthetic device that may be fabricated using a low-cost 3D printer. Grasp experiments may be performed and the successful grasps are shown in FIGS. 17(A) and 17(B). According to the prosthetic hand of some exemplary embodiments, the prosthetic hand can perform 31 out of the 33 grasps. For example, the grasps may include power, intermediate and precision type grasps with thumb abductions and adductions. The grasps may include grasps of objects like coins, credit cards, plates, bottles, can, tissue rolls, pens, markers, and so on. Further, as shown in FIG. 18, the prosthetic hand of some exemplary embodiments may be used to perform social gestures, such as handshake and victory-sign pose.

The ability to obtain accurate anthropometric data from the human hand may be applicable to numerous fields. The design of tools and devices may use the collection of hand measurements from individuals or large groups of people. Procedures according to various exemplary embodiments discussed herein may enable the extraction of required measurements in an inexpensive and controlled manner therefore has many commercial uses. Exemplary procedures can be performed manually by visual identification of natural landmarks and/or automated through the implementation of computer vision techniques.

Consequently, the procedures and devices of various exemplary embodiments may be of interest to companies designing devices, such as personalized upper-limb medical devices, gloves, or ergonomic handheld tools, as well as individuals and companies interested in developing software to automate the process may also hold interest. Automation of such exemplary procedures may enable acquisition of large amounts of anthropometric hand data, which may facilitate research applications or the development of neural models to perform generalizations and predictions.

To verify the commercialization potential of this method, certain embodiments utilized a procedure to calculate relevant anthropometric measures of the non-affected hands of four individuals with missing hands. These measures were then used to make recommendations for the appropriate model of prosthetic gloves from manufacturers. The four subjects (3 females, 1 male, 8-21 years old) were recruited from a field work in Jordan. Two of the participants have missing part of the upper limb below the elbow while the other two have missing part of the upper limb above the elbow.

With 3D scanning, various exemplary embodiments may have implemented a joint-center procedure (s) for the selection of prosthetic gloves for the participants. From the interviews that were conducted with the participants, it was learned that lifelike appearance of a prosthetic hand may be important. This feedback was not surprising as earlier works on patient preferences indicate that users expect a prosthetic hand or finger to be of an appropriate size and weight, as well as lifelike in appearance to ward off psychosocial stigma. The prosthetic gloves, which are available from manufacturers, may be designed to be integrated to the prosthetic hands as a covering. The prosthetic arms may then be attached to the stump of the missing limb through, e.g., a socket.

For the exemplary experiment described herein, three product segments from leading manufacturers of passive prosthetic hands or gloves were selected. These manufacturers provided guidance on where the measurements are to be taken. Once the measurements were obtained, a corresponding model of the prosthetic glove may be determined from a given set of tables shown in Table VII below.

TABLE VII
Suggested prosthetic glove models

| Manufacturer | Participant 1 | Participant 2 | Participant 3 | Participant 4 |
|---|---|---|---|---|
| Ottobock | 8S6 = 151 × 58L | 8S5 = 174 × 74L | 8S5 = 167 × 72R | 8S4 = 230 × 93R |
| Regal | 102L-F-L-CXS | 102L-F-L-S1 | 102L-F-R-S1 | 102L-M-R-L |
| Steeper | B11079 | B11075 | B11077 | B11060 |

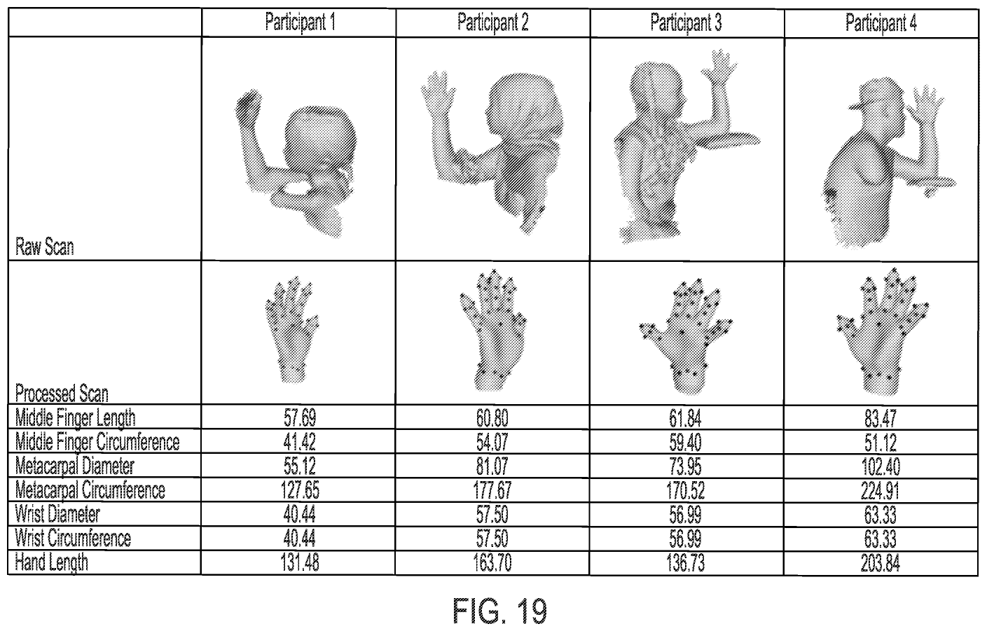

The raw and the processed scans of the participants' non-affected hands are shown in FIG. 19. To select the prosthetic glove for the missing hand, the various measurements that may be needed for the selection are also shown. All of these measurement variables may be needed to make the selection.

As an example shown in FIG. 19, Ottobock may require the middle finger length and the metacarpal circumference, while the other manufacturers may require more variables to be measured. Table VII shows the suggested models of prosthetic glove from various manufacturers.

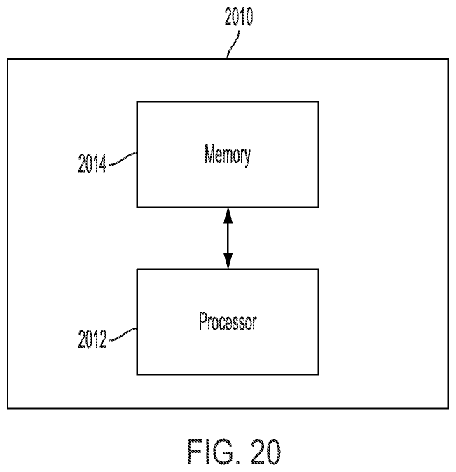

As shown in FIG. 20, various exemplary embodiments may provide an apparatus 2010, such as a computer or other computational device, medical device, or computer system or network. The apparatus 2010 may include at least one processor 2012, which may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. While a single processor may be discussed and shown in FIG. 20, multiple processors may be utilized according to other exemplary embodiments. For example, it should be understood that, in certain exemplary embodiments, apparatuses 2012 may include two or more processors that may form a multiprocessor system that may support multiprocessing.

The apparatus 2010 may also include at least one memory storing instructions that, when executed by the at least one processor 2012, cause the apparatus 2010 to perform one or more methods, procedures, and/or processes according to various exemplary embodiments discussed herein.

The memory 2014 may be internal or external to the apparatus 2012 and may be coupled to the processor 2012 for storing information and instructions. Memory 1014 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 2014 can be comprised of any combination of random access memory (RAM), read only memory (ROM), static storage such as a magnetic or optical disk, hard disk drive (HDD), or any other type of non-transitory machine or computer readable media.

According to certain exemplary embodiments, the apparatus 2010 may be caused, by the processor 2012 executing the instructions stored in the memory 2014, to at least receive, from one or more measurement devices, one or more scans of a hand of a subject, and identify a plurality of landmark positions on the hand by performing an imaging analysis of the one or more scans. The apparatus 2010 may also be caused to determine a plurality of measurements related to one or more of a plurality of fingers of the hand based on one or more of the identified landmark positions and generate a point cloud of the plurality of determined measurements.

Some exemplary embodiments may provide that the apparatus 2010 may be further caused to generate a three-dimensional computer model of the hand based on the generated point cloud of the plurality of measurements, and to output the generated three-dimensional computer model of the hand to a three-dimensional printing device configured to manufacture a prosthetic hand corresponding to the generated three-dimensional computer model of the hand.

According to various exemplary embodiments, the plurality of landmark positions may include a center of each of a plurality of joints of the plurality of fingers of the hand. Further, the plurality of landmark positions may be used to determine at least one of: measurements representing finger lengths based on the center of each of the plurality of joints, measurements representing wrist and palm circumferences, and/or measurements representing palm length, which is a vertical distance between a specified center of one of the plurality of joints and a center of the wrist.

Various exemplary embodiments may also provide a method performed by an apparatus, such as apparatus 2010. The method may include receiving, from one or more measurement devices, one or more scans of a hand of a subject, and identifying a plurality of landmark positions on the hand by performing an imaging analysis of the one or more scans. The method may further include determining a plurality of measurements related to one or more of a plurality of fingers of the hand based on one or more of the identified landmark positions and generating a point cloud of the plurality of determined measurements.

Some exemplary embodiments may also provide that the method may include generating a three-dimensional computer model of the hand based on the generated point cloud of the plurality of measurements. Further, certain exemplary embodiments may provide that the method includes outputting the generated three-dimensional computer model of the hand to a three-dimensional printing device configured to manufacture a prosthetic hand corresponding to the generated three-dimensional computer model of the hand.

According to certain exemplary embodiments, the plurality of landmark positions may include a center of each of a plurality of joints of the plurality of fingers of the hand. Further, the plurality of landmark positions may be used to determine at least one of: measurements representing finger lengths based on the center of each of the plurality of joints, measurements representing wrist and palm circumferences, and/or measurements representing palm length, which is a vertical distance between a specified center of one of the plurality of joints and a center of the wrist.

From anthropological studies as well as interviews with prosthesis users, including the aforementioned subjects, it may be understood that a passive cosmetic prosthesis may be useful for many amputees and people with congenital defects. Of the participants in the experiment of FIG. 19, four of them were born with congenital upper limb reductions and the fifth one lost their hand and leg in a war. One young patient said that she goes to school and wanted a prosthetic that could help her with lightweight activities such as holding a phone, mirror, or makeup box. She could then use her healthy hand to operate the phone or apply the makeup. She also said that she wants a prosthetic that looks realistic to facilitate social interactions without her disability or prosthesis bringing unwanted attention. She admitted that a cosmetic prosthetic would improve her self-esteem and body-image and help her feel better about herself and her body. Her story was inspiring, and the other patients shared similar sentiment about their prostheses. Their stories inspired the inventors to explore and innovate on the passive prosthesis and develop this design.

Furthermore, many children are also born without limbs due to congenital defects. They need special care to accommodate them and support their education and development through alternative means. Due to long-lasting wars in many parts of the world, other people lose their limbs. Many of these amputees suffer from trauma in addition to their physical disability. Prosthetic users, especially children, and adolescents need frequent adjustments as they grow. Adults need prosthetics with functionality that allow them to live with dignity and enable them to return to work.

The features, structures, or characteristics of exemplary embodiments described throughout this specification may be combined in any suitable manner in one or more exemplary embodiments. For example, the usage of the phrases “certain embodiments,” “an exemplary embodiment,” “some embodiments,” or other similar language, throughout this specification refers to the fact that a particular feature, structure, or characteristic described in connection with an embodiment may be included in at least one embodiment. Thus, appearances of the phrases “in certain embodiments,” “an exemplary embodiment,” “in some embodiments,” “in other embodiments,” or other similar language, throughout this specification do not necessarily refer to the same group of embodiments, and the described features, structures, or characteristics may be combined in any suitable manner in one or more exemplary embodiments.

As used herein, “at least one of the following: <a list of two or more elements>” and “at least one of <a list of two or more elements>” and similar wording, where the list of two or more elements are joined by “and” or “or,” mean at least any one of the elements, or at least any two or more of the elements, or at least all the elements.

One having ordinary skill in the art will readily understand that the disclosure as discussed above may be practiced with procedures in a different order, and/or with hardware elements in configurations which are different than those which are disclosed. Therefore, although the disclosure has been described based upon these exemplary embodiments, it would be apparent to those of skill in the art that certain modifications, variations, and alternative constructions would be apparent, while remaining within the spirit and scope of the exemplary embodiments or the invention as disclosed.

We claim:

1. An apparatus, comprising:
at least one processor; and
at least one memory storing instructions that, when executed by the at least one processor, cause the apparatus at least to:
receive, from one or more measurement devices, one or more scans of a hand of a subject;
identify a plurality of landmark positions on the hand by performing an imaging analysis of the one or more scans;
determine a plurality of measurements related to one or more of a plurality of fingers of the hand based on one or more of the identified landmark positions; and
generate a point cloud of the plurality of determined measurements.

2. The apparatus according to claim 1, wherein the apparatus is further caused to:
generate a three-dimensional computer model of the hand based on the generated point cloud of the plurality of measurements.

3. The apparatus according to claim 2, wherein the apparatus is further caused to:
output the generated three-dimensional computer model of the hand to a three-dimensional printing device configured to manufacture a prosthetic hand corresponding to the generated three-dimensional computer model of the hand.

4. The apparatus according to claim 1, wherein the plurality of landmark positions include a center of each of a plurality of joints of the plurality of fingers of the hand.

5. The apparatus according to claim 4, wherein the plurality of landmark positions are used to determine at least one of:
measurements representing finger lengths based on the center of each of the plurality of joints;
measurements representing wrist and palm circumferences; or
measurements representing palm length, which is a vertical distance between a specified center of one of the plurality of joints and a center of the wrist.

6. A method of calculating features of a prosthetic hand, comprising:
receiving, from one or more measurement devices, one or more scans of a hand of a subject;
identifying a plurality of landmark positions on the hand by performing an imaging analysis of the one or more scans;
determining a plurality of measurements related to one or more of a plurality of fingers of the hand based on one or more of the identified landmark positions; and
generating a point cloud of the plurality of determined measurements.

7. The method according to claim 6, further comprising:
generating a three-dimensional computer model of the hand based on the generated point cloud of the plurality of measurements.

8. The method according to claim 7, further comprising:
outputting the generated three-dimensional computer model of the hand to a three-dimensional printing device configured to manufacture a prosthetic hand corresponding to the generated three-dimensional computer model of the hand.

9. The method according to claim 6, wherein the plurality of landmark positions include a center of each of a plurality of joints of the plurality of fingers of the hand.

10. The method according to claim 9, wherein the plurality of landmark positions are used to determine at least one of:
measurements representing finger lengths based on the center of each of the plurality of joints;
measurements representing wrist and palm circumferences; or
measurements representing palm length, which is a vertical distance between a specified center of one of the plurality of joints and a center of the wrist.

11. An apparatus, comprising:
a body frame of a prosthetic hand; and
a plurality of finger frames connected to the body frame, each of the plurality of finger frames being formed of multiple segments connected by ball and socket joints,
wherein the plurality of finger frames are configured to bend and rotate relative to the joints and to maintain a position in which the finger frames are positioned.

12. The apparatus according to claim 11, wherein the plurality of finger frames are moved to a position by an external force and maintain the position such that the apparatus is configured to grasp an object.

13. The apparatus according to claim 11, wherein:
the multiple segments of each of the plurality of fingers is at least a proximal segment, a middle segment, and a distal segment; and
the proximal segment is connected to the body frame by one ball and socket joint.

14. The apparatus according to claim 11, wherein the body frame and the plurality of fingers are formed of acrylonitrile butadiene styrene or polylactic acid.
